(19) United States
(12) Patent Application Publication
(10) Pub. No.: US 2011/0059035 A1
(43) Pub. Date: Mar. 10, 2011

(54) COMPOSITIONS BASED ON POLYMER/HYDROGEN PEROXIDE COMPLEXES AND USES THEREOF

(75) Inventors: Anne-Laure Bernard, Antony (FR); Dominique Bordeaux, Soisy Sur Seine (FR)

(73) Assignee: L"OREAL

(21) Appl. No.: 12/990,234

(22) PCT Filed: Apr. 30, 2009
(86) PCT No.: PCT/FR09/50807

§ 371 Date: Oct. 29, 2010

Related U.S. Application Data
(60) Provisional application No. 61/054,827, filed on May 21, 2008.

(30) Foreign Application Priority Data
Apr. 30, 2008 (FR) 0852951

(51) Int. Cl. A61K 8/81; A61Q 19/02; A61Q 19/08; A61Q 19/00; A61Q 1/00

(52) U.S. Cl. 424/62; 424/78.03

(57) ABSTRACT

The invention relates to a cosmetic assembly for improving the appearance of the skin, comprising a first anhydrous composition comprising a polymer-H₂O₂ complex, said polymer having hydrogen-bond acceptor groups which are capable of forming a noncovalent chemical bond with hydrogen peroxide, and a second composition comprising an activator which promotes the decomposition of H₂O₂.

The invention relates to a cosmetic assembly comprising a first anhydrous composition comprising a polymer-H₂O₂ complex and a second composition comprising an activator which promotes the decomposition of H₂O₂.

It also relates to the use of this cosmetic assembly in the field of cosmetics or dermatology or to a cosmetic method comprising the application of this assembly to the skin.

Consumers are looking for skin care products having a measurable short-term effectiveness with a noticeable and lasting improvement in the appearance of the skin. In particular, the desired effects are a smoothing of the skin relief, pores which are less apparent and/or a microrelief and wrinkles which are less visible, a spot attenuation, a more radiant complexion, in particular for dull skin or dark skin, or skin which is cleaner or less shiny, in particular for greasy skin. These results can be obtained with conventional care products over relatively long treatment times (greater than 3 months). Other treatments, such as chemical peelings, also make it possible to improve the appearance of the skin in a lasting and perceptible fashion, after several application sessions. There are generally major disadvantages to these treatments, such as, for example, significant desquamation a few days after an application session.

Greasy skin is characterized by a skin which is shining, sometimes with an oily appearance, which is thick and which has dilated pilosebaceous pores. Sebaceous hypersecretion is generally related to hyperandrogenism due either to excessive production of androgens by an endocrine gland or to excessive peripheral production in the sebaceous gland from the surrounding proandrogens and/or androgens.

Greasy skin having a tendency towards acne generally exhibits skin imperfections, dilated pores, a nonuniform texture of the skin and red patches. However, the known compositions do not make it possible to rapidly obtain a noticeable and lasting improvement in the appearance of the skin. Furthermore, it would be desirable to have available care products for the skin which are more stable and better tolerated than the known compositions of the prior art.

Formulations comprising polyvinylpyrrolidone-H₂O₂ complexes have been proposed for the treatment of acne. Thus, U.S. Pat. No. 5,122,370 (ISP) describes the use of polyvinylpyrrolidone-H₂O₂ complexes dispersed in an anhydrous medium for the treatment of acne. However, the formulations proposed are not satisfactory for a cosmetic use such as envisaged in the present invention.

The need thus remains to provide such cosmetic treatments for the noticeable and lasting improvement in the appearance of the skin, in particular for the purpose of obtaining a smoothing of the skin relief, pores which are less apparent and/or a microrelief and wrinkles which are less visible, a spot attenuation, a more radiant complexion, in particular for dull skin or dark skin, and skin which is cleaner or less shiny, in particular for greasy skin.

The Applicant has been able to show, surprisingly and unexpectedly, that a cosmetic assembly as defined below makes it possible to meet the needs specified above.

The compositions of the cosmetic assembly prepared by the Applicant are stable and particularly effective for improving the appearance of the skin. Furthermore, the care provided with the cosmetic assembly according to the present invention is better tolerated by the skin than the available cosmetic care.

A subject matter of the invention is thus a cosmetic assembly comprising:
a first anhydrous composition comprising at least one polymer-H₂O₂ complex, said polymer exhibiting hydrogen bond-acceptor groups capable of forming a noncovalent chemical bond with hydrogen peroxide; and
a second composition comprising at least one activator which promotes the decomposition of H₂O₂.

The use of the cosmetic assembly according to the invention makes it possible to improve the appearance of the skin, in particular by attenuating the imperfections of the skin, spots and acne lesions, by smoothing the skin relief, by rendering the pores less apparent (in particular by reducing the size of the pores), by reducing the number of pimples and blackheads, by rendering the microrelief and the wrinkles less visible, by restoring radiance to the skin, in particular dull, ashen or dark skin, and/or by improving the appearance of greasy skin, in particular by promoting a matt appearance of the skin or by reducing skin shining. The cosmetic assembly according to the invention also makes it possible to lighten the skin, to promote the radiance of the complexion, to render the complexion uniform, to reduce spots, in particular age spots or blotches due to the sun, or to improve the hold of makeup.

An object of the invention is thus a cosmetic method for improving the appearance of the skin and in particular for giving at least one of the abovementioned effects to the skin, comprising the application, to the skin, of the cosmetic assembly according to the invention, after mixing the compositions constituting said assembly.

Another object of the invention is a cosmetic care method of greasy skin, in particular for the purpose of rendering the skin matt, comprising the topical application, on the skin, of the cosmetic assembly as described above, after mixing the compositions constituting said assembly.

The compositions constituting the cosmetic assembly according to the invention will now be described in detail.

The first composition of the cosmetic assembly according to the invention is thus an anhydrous composition which comprises at least one polymer-H₂O₂ complex.

The term “polymer-H₂O₂ complex” in the context of the present invention denotes a complex between a polymer as defined below and hydrogen peroxide. The complex can in particular be formed before it is incorporated in the first composition according to the invention. In said complex, the polymer and hydrogen peroxide are noncovalently bonded, in particular via hydrogen bonds.

The term “polymer” denotes, in the context of the present invention, any polymer exhibiting groups which are hydrogen bond-acceptor groups which are capable of forming a noncovalent chemical bond with hydrogen peroxide. The hydrogen bond-acceptor groups are, in the linear structure (or linear main chain) of said polymer, advantageously separated by a distance of less than or equal to 5 carbon atoms, preferably of less than or equal to 3 carbon atoms, so as to form stable hydrogen bonds with hydrogen peroxide. Preferably, hydrogen bond-acceptor groups form noncovalent chemical bonds with hydrogen peroxide in a molecular ratio of 1:1, of 2:1 or a mixture of these ratios. Hydrogen bond-acceptor groups form part of the general knowledge in the field of chemistry. Mention may be made of heteroatoms carrying free electron pair(s), such as oxygen or nitrogen atoms.

Hydrogen bond-acceptor groups are preferably N—C═O or N—C═N groups, which can be incorporated in the linear structure of said polymer or carried by a carbon-comprising side chain of the polymer, for example a linear chain or a ring, preferably a ring.

Preferably, the polymer is capable of forming a continuous film on the skin, thus exhibiting a film-forming nature.

Preference is given, among the polymers which can be used in the first composition of the cosmetic assembly according to the invention, to a polymer based on N-vinylheterocyclic units.

Polymers based on N-vinylheterocyclic units are homo- or copolymers deriving from N-vinylheterocyclic monomers, preferably comprising N-vinylheterocyclic monomers exhibiting from 3 to 7 atoms in the heterocyclic ring, including a carbonyl (C═O) group and a nitrogen atom, and comprising a vinyl group. Preferably, the ring comprises 5 or 6 atoms and comprises heteroatoms such a sulfur or oxygen, and may be substituted or not. Preferably, the N-vinylheterocyclic monomers are N-vinylcaprolactam, N-vinylpyrrolidone or N-vinylimidazole or mixtures thereof. The comonomers which can be envisaged can be vinyl acetate, isopropenyl acetate, vinyl laurate, vinyl benzoate, methyl vinyl ether, 2-methylvinylimidazole, N-vinylpiperidone, N-vinyl-2-oxazolidone, methyl acrylate, ethyl acrylate, styrene or a cationically modified vinyl monomer. The appropriate copolymers comprise, for example, from 30 to 99% by weight of N-vinyl monomers and from 1 to 70% by weight of a comonomer, with respect to the total weight of the polymer. Advantageously, the polymer is poly-N-vinyl-poly-2-pyrrolidone or one of its copolymers. Poly-N-vinyl-poly-2-pyrrolidone is also commonly known as polyvinylpyrrolidone or “PVP”. The polymers include soluble or nonsoluble, crosslinked or not, PVP polymers. Copolymers that contain PVP can derive from vinylpyrrolidone/vinyl acetate (also known under the name copolyvidone, copolyvidonum or VP-VAc) or from vinylpyrrolidone/dimethylaminoethyl methacrylate.

In the context of the present invention, the term “polyvinylpyrrolidone-H₂O₂ complex” or “PVP-H₂O₂” denotes a complex between a polyvinylpyrrolidone as defined above and hydrogen peroxide. PVP-H₂O₂ complexes have as advantage their film-forming properties and the fact that they exhibit the same activity as free hydrogen peroxide, at an equivalent level of H₂O₂, whatever the formulation medium is. They make possible easy application and good hold of the product on the face while avoiding it flowing into the eyes, in particular. Polymer-H₂O₂ complexes can be produced by virtue of procedures well known to a person skilled in the art.

In particular, one of the methods described in U.S. Pat. No. 5,077,047 (ISP), U.S. Pat. No. 5,122,370 (ISP) or U.S. Pat. No. 5,674,436 (BASF), and in the references cited in these patents, can be chosen. Alternatively, commercially available polymer-H₂O₂ complexes will be chosen. Mention may be made, on this account, of the products sold by ISP under the Peroxydone® brand, in particular Peroxydone® K-30 and K-90 products, preferably Peroxydone® K-30. Peroxydone® K-30 and K-90 products are linear homopolymers of N-vinylpyrrolidone with molecular weights of 58 000 and 1 300 000 respectively complexed with hydrogen peroxide, present in an amount of 17 to 20% by weight with respect to the total weight of the complex.

The amount of hydrogen peroxide in the complex described above can be expressed as percentage by weight of hydrogen peroxide with respect to the total weight of said complex.

Peroxide index (PI) is the amount of product (the polymer-H₂O₂ complex comprising the hydrogen peroxide) present in the sample, expressed as milliequivalents of active oxygen per kilogram, which oxidizes potassium iodide under the operating conditions defined by the standard ISO 3960 and AFNOR T 60-220.

The percentage by weight of H₂O₂ in the complex corresponds to:

PI of the association comprising the peroxide/1000/10×PM,
with:
MW=molecular weight of H₂O₂ and
PI=peroxide index for a raw material (in particular the polymer-H₂O₂ complex comprising the hydrogen peroxide) calculated under the operating conditions defined by the standard ISO 3960 and AFNOR T 60-220.

By way of example, the amount of hydrogen peroxide in said complex can in particular be between 0.1 and 30% by weight, preferably 10 and 25% by weight, with respect to the weight of said complex.

Preferably, the amount of H₂O₂ in the first composition is between 0.1 and 30%. Preferably, it is between 0.1 and 25% by weight, preferably between 0.1 and 20% by weight.

In a preferred embodiment, the first composition of the cosmetic assembly comprises from 0.5 to 25% by weight of hydrogen peroxide, with respect to the total weight of said first composition.

Advantageously, the amount of complex described above is such that the content of H₂O₂ in the cosmetic assembly (that is to say, the composition resulting from the mixing of the first and second compositions of the cosmetic assembly defined above) is between 0.01 and 7% by weight, preferably between 0.05 and 5% by weight, preferably between 0.1 and 2% by weight, with respect to the total weight of the cosmetic assembly, specific contents of H₂O₂ being 0.05, 0.1, 0.2, 0.3, 0.4, 0.5, 0.6, 0.7, 0.8, 0.9, 1, 2, 3 and 4% by weight, with respect to the total weight of the cosmetic assembly.

The amount of polymer-H₂O₂ complex in the first composition can be in particular between 0.1 and 100% by weight, with respect to the total weight of the first composition.

According to a specific form, the first composition consists of the polymer-H₂O₂ complex, said polymer preferably being PVP. According to this form, the first composition is in particular in form of a powder.

According to another embodiment, the first composition comprises in particular between 0.5 and 85% by weight of the polymer-H₂O₂ complex, in particular between 1 and 75% by weight, between 2 and 55% by weight, between 3 and 45% by weight, preferably between 5 and 40% by weight, and still more preferably between 10 and 40% by weight, with respect to the total weight of the first composition. Advantageously, the complex is present in an amount of between 11 and 22% by weight, with respect to the total weight of the first composition.

According to a specific embodiment of the invention, the first composition of the cosmetic assembly comprises a polymer-H₂O₂ complex as defined above and an anhydrous vehicle which is acceptable cosmetically or dermatologically. As the assembly according to the invention is intended for topical use on the skin, the vehicle in the first composition must be acceptable cosmetically or dermatologically, more particularly without unpleasant odor, color or appearance, and which does not generate tingling, tightening or redness unacceptable to the user.

The term “vehicle which is acceptable cosmetically or dermatologically” is understood to mean a medium compatible with the keratinous substances of human beings, such as skin, mucous membranes, nails, scalp and/or hair.

The term “vehicle” in the context of the present invention denotes an organic solvent or a mixture of organic solvents. The organic solvents can be selected in particular from vehicles based on glycols (glycol vehicles), alcohols (alcohol vehicles), ketones, ethers, esters, aldehydes and their mixture, which are acceptable cosmetically or dermatologically.

The term “alcohol vehicle” is understood to mean a vehicle comprising at least one water-soluble lower monoalcohol representing at least 50% by weight of the vehicle. The term “lower monoalcohol” is understood to mean an alcohol comprising from 1 to 8 carbon atoms and comprising only a single hydroxyl functional group. Mention may be made, as lower monoalcohols, for example, of ethanol, isopropanol, butanol, pentanol and their mixtures. Preferably, primary alcohols comprising from 1 to 4 carbon atoms are chosen. Ethanol is preferred.

The term “glycol vehicle” is understood to mean a vehicle comprising at least one organic compound exhibiting at least two hydroxyl functional groups (named, in the present description, glycol).

Mention may in particular be made of:
glycerol;
monomeric glycols and in particular diols, such as especially propylene glycol, dipropylene glycol, butylene glycol, diethylene glycol, ethylene glycol, hexylene glycol, isoprene glycol, pentylene glycol, butanediol, 1,2-propanediol, 1,3-propanediol, pentanediols, in particular 1,5-pentanediol, decanediol, dodecanediol and their ethers, such as propylene glycol monomethyl ether, dipropylene glycol monobutyl ether or 2-butoxyethanol;
polymeric glycols, such as, in particular, high- or low-molecular-weight polyethylene glycols (PEGs) and especially polyethylene glycols with a molecular weight of less than 600, such as PEG-8 (or Polyethylene 400), PEG-6 and PEG-12.

Mention may be made, among preferred glycols, of polymeric glycols, in particular polyethylene glycols with a molecular weight of less than 600, such as PEG-8 (or Polyethylene 400), PEG-6 and PEG-12.

Mention will be made of PEG-8 or PEG-6 as preferred PEG.

According to a specific form, the glycol vehicle according to the invention comprises at least two glycols selected from monomeric glycols and PEGs, or low-molecular-weight PEGs and high-molecular-weight PEGs.

According to another specific form, the glycol vehicle according to the invention comprises at least one diol and at least one glycol chosen from glycerol or a PEG. By way of example, the glycol vehicle can comprise the following combinations of glycols:
(propylene glycol or butylene glycol) and glycerol;
(propylene glycol or butylene glycol) and PEG-8;
(propylene glycol or butylene glycol) and glycerol and PEG-8.

Advantageously, the glycol vehicle according to the invention comprises butylene glycol and optionally PEG-8. Preferably, the glycol vehicle comprises PEG-8.

In a preferred embodiment, the first composition of the cosmetic assembly according to the invention comprises a PVP-H₂O₂ complex and a glycol vehicle comprising PEG-8.

According to a preferred form, when the glycol vehicle comprises a glycols/glycerol mixture, the glycerol content will be less than 50% by weight of glycerol, with respect to the total weight of the vehicle.

According to another preferred form, when the vehicle is composed of a mixture of a glycol vehicle based on glycerol and of an alcohol vehicle (in particular based on ethanol), glycerol content will be less than 50% by weight of glycerol, with respect to the total weight of the vehicle.

According to another form, the vehicle comprises a vehicle comprising at least one glycol as defined above (including the preferred forms), preferably a monomeric glycol and/or a polymeric glycol, and at least one alcohol as defined above, preferably ethanol.

Mention may in particular be made, among the other organic solvents which may be of use as vehicles in the composition, of: ketones which are liquid at ambient temperature (20-25° C.), such as methyl ethyl ketone, methyl isobutyl ketone, diisobutyl ketone, isophorone, cyclohexanone, acetone; propylene glycol ethers which are liquid at ambient temperature, such as propylene glycol monomethyl ether, propylene glycol monomethyl ether acetate, dipropylene glycol mono(n-butyl)ether; cyclic ethers, such as gamma-butyrolactone; short-chain esters (having from 3 to 8 carbon atoms in total), such as ethyl acetate, methyl acetate, propyl acetate, isopropyl acetate, n-butyl acetate, isopentyl acetate, methoxypropyl acetate or butyl lactate; ethers which are liquid at ambient temperature, such as diethyl ether, dimethyl ether or dichlorodiethyl ether; aldehydes which are liquid at ambient temperature, such as benzaldehyde or acetaldehyde; and their mixtures.

In a particularly preferred embodiment, the vehicle of the first composition comprises a low amount of oil. In other words, the amount of oil generally does not exceed 10% by weight, with respect to the total weight of the first composition. The amount of oil in the first composition is preferably less than or equal to 7% by weight, with respect to the total weight of the first composition, even more preferably less than 5% by weight, preferably less than 4% by weight. Preferably, the vehicle of the first composition does not comprise oil. As indicated above, the polymers used in the complex of the first composition preferably have film-forming properties. Consequently, the mixture of the film-forming polymer with an oil, if the oil is in a higher amount than indicated above, results in the formation of a composition exhibiting an appearance and a texture not very compatible with a cosmetic use. The term “oil” is understood to mean, according to the invention, a fatty substance which is liquid at ambient temperature (approximately 25° C.) and in particular as defined below.

In a particularly advantageous embodiment, the first composition according to the present invention comprises, as polymer-H₂O₂ complex, one of the PVP-H₂O₂ complexes sold by ISP under the name Peroxydone®, in particular the products Peroxydone® K-30 and K-90, preferably Peroxydone® K-30. Preferably, this PVP-H₂O₂ complex is dispersed in PEG-8.

The first composition of the cosmetic assembly according to the invention is anhydrous. In other words, the water content in the first composition is low. Thus, the amount of water generally does not exceed 10% by weight, with respect to the total weight of the first composition. It can in particular comprise a small amount of water which generally originates from the raw materials used. The amount of water in the first composition is preferably less than or equal to 7% by weight, with respect to the total weight of the first composition, even more preferably less than 5% by weight, preferably less than 4% by weight. The low content of water in the first composition according to the invention has as effect a greater stability of the hydrogen peroxide in the composition.

In a preferred embodiment, the first composition of the assembly according to the invention does not comprise an activator which promotes the decomposition of H₂O₂ (see below the description of the second composition for the definition of the term “activator which promotes the decomposition of H₂O₂”). The activator is thus separated from hydrogen peroxide, which makes it possible to improve the effectiveness of the cosmetic care according to the invention.

The second composition of the cosmetic assembly comprises at least one activator which promotes the decomposition of H₂O₂.

The term “activator which promotes the decomposition of H₂O₂” covers any molecule or composition which can result in the decomposition of H₂O₂ into water and oxygen.

This term thus covers a large number of compounds known to a person skilled in the art. Mention may in particular be made, as activators, of basic compounds, especially sodium or potassium hydroxide hydroxide, ammonia or triethanolamine, sodium (or ammonium) bicarbonate, sodium carbonate, manganese gluconate, sodium borate, calcium peroxide, calcium hydroxide, basic amino acids and their salts, such as lysine, arginine and their salts; guanidine; potassium carbonate, urea, aminomethylpropanol, aminopropanol, monoethanolamine (MEA), aminopropyltriethoxysilane (APTES), sodium or magnesium silicate derivatives, in particular laponite (especially Laponite XLG, sold by Rockwood), smectite, sodium metasilicate, hectorite, basic zeolites, in particular the zeolite sold by Zeochem under the reference X-MOL, or their mixture. Advantageously, the activators are selected from sodium bicarbonate, laponite, zeolites, calcium peroxide, sodium hydroxide (preferably at 10% in water), sodium metasilicate and their mixture. The activators are preferably selected from sodium bicarbonate, zeolites and calcium peroxide. Particularly preferably, the activator is a zeolite. Thus, in a specific embodiment, the second composition of the cosmetic assembly according to the invention comprises a zeolite.

Advantageously, the cosmetic assembly according to the invention comprises
a first anhydrous composition comprising a PVP-H₂O₂ complex, preferably in a glycol vehicle comprising PEG-8, and
a second composition comprising a zeolite.

The activator can preferably be selected from the compounds or compositions capable of generating a pH of greater than 8.5, preferably greater than 9, when they are mixed with a polymer-H₂O₂ complex at 11% by weight in water (with a percentage of H₂O₂ of 2% in the aqueous composition), with respect to the total weight of the mixture (polymer-H₂O₂/water).

In the case of a PVP-H₂O₂ complex, the following protocol can be used to select the activators. A dispersion of 11 g of Peroxydone K-30® (ISP) in 75 g of water is produced, to which increasing amounts of activator are added. pH is measured immediately after each addition. The activator is added in excess until the activator/H₂O₂ ratio by weight reaches 10. In this example, the amount of H₂O₂ is approximately 2 g as Peroxydone K-30® comprises 18.5% of H₂O₂ by weight, with respect to the total weight of Peroxydone K-30® (that is to say, of the complex).

The preferred activators are selected from laponite XLG from Rockwood, zeolite X-MOL sold by Zeochem, calcium peroxide, sodium hydroxide (at 10% in water), sodium metasilicate and their mixture (cf. table 1 below).

TABLE 1

| | Minimum amount in | |
|---|---|---|
| | order to reach a pH > 9 or | |
| | a plateau pH if pH < 9 | |
| | (for an initial amount of | |
| | 11 g of Peroxydone K-30 ®, | |
| Activator | i.e. 2 g of H₂O₂) | Associated pH |
| Sodium bicarbonate | 0.9 g | 8.1 |
| Zeolite | 4.2 g | 9.1 |
| Laponite | 3 g | 9.3 |
| Calcium peroxide | 0.05 g | 9.3 |
| 10% NaOH in water | 0.19 g | 9.3 |
| Sodium metasilicate | 0.11 g | 9.8 |

A person skilled in the art will take particular care to introduce a sufficient amount of activator for the decomposition of H₂O₂ to be effective in improving the appearance of the skin of the user, after mixing the compositions of the cosmetic assembly according to the invention.

The choice will preferably be made of one or more activators which make it possible to obtain, in the test described above, a pH of between 9 and 11, preferably between 9 and 10.

The amount of activators in the second composition depends on the components of the first and second compositions of the assembly according to the invention, on their respective amounts and on their galenic forms. In particular, a person skilled in the art will take care to choose the amount of activators so that, when the compositions of the cosmetic assembly according to the invention are mixed, the mixture resulting therefrom can express a pH of greater than 8.5, preferably of greater than 9, and in particular between 9 and 11, preferably between 9 and 10. For tolerance reasons, however, a pH of between 9 and 9.5 will be preferred. By way of illustration, use may be made of amounts of activators defined on the basis of the above table 1.

According to a specific form, the pH of the mixture will be between pH 8.3 and 9 inclusive.

The term “possibility of expressing a pH” in the context of the present invention is understood to define the pH of the mixture of the two compositions of the cosmetic assembly when this mixture comprises water.

The first composition according to the invention is anhydrous and the second composition may possibly be anhydrous also. In this case, the mixture of the two anhydrous compositions does not make it possible to measure a pH. In order to know what is the pH expressed by the anhydrous mixture, water is added to this mixture, for example in an amount of greater than 15%, preferably of greater than 20% by weight, with respect to the water+mixture of the compositions total weight, and the pH is measured. This measured pH is the expressed pH.

The second composition comprises at least one activator and optionally a vehicle which is acceptable cosmetically or dermatologically.

According to an alternative form, the second composition consists solely of one or more activators. According to this alternative form, the second composition can be in a powder form. According to a preferred embodiment of this alternative form, the activator is a zeolite in a powder form. Even more preferably, the first composition comprises a PVP-H₂O₂ complex and a glycol vehicle comprising PEG-8 and the second composition is composed of a zeolite in powder form.

According to another alternative form, the second composition comprises at least one activator and a vehicle which is cosmetically or dermatologically acceptable. As the assembly according to the invention is intended for topical use on the skin, the vehicle in the second composition must also be acceptable cosmetically or dermatologically, more particularly without unpleasant odor, color or appearance, and which does not generate tingling, tightening or redness unacceptable to the user.

The same vehicles as those described above for the first composition of the cosmetic assembly can be used in the second composition. In addition, the second composition can comprise at least one oil as vehicle. It can also comprise water.

Mention may be made, for example, of oils which can be used in the composition of the invention, of:
linear or branched hydrocarbons of mineral or synthetic origin, such as volatile or nonvolatile liquid paraffins and their derivatives, petrolatum, polydecenes or hydrogenated polyisobutene such as Parleam® oil; branched C₈-C₁₆ alkanes, such as C₈-C₁₆ isoalkanes, such as isododecane (also known as 2,2,4,4,6-pentamethylheptane), isodecane or isohexadecane;
triglycerides, such as liquid triglycerides of fatty acids comprising from 4 to 10 carbon atoms, such as triglycerides of heptanoic acid or octanoic acid; or also hydrocarbon oils of vegetable origin, such as, for example, sweet almond, sunflower, corn, soybean, squash, coriander, grape seed, sesame, hazelnut, apricot (Prunus armenica Oil), macadamia, arara, castor or avocado oils, triglycerides of caprylic/capric acids such as those sold by Stearineries Dubois or those sold under the names Miglyol 810, 812 and 818 by Dynamit Nobel, jojoba oil or shea butter oil;
synthetic esters and ethers, in particular of fatty acids, such as oils of formulae R¹COOR² and R¹OR² in which R¹ represents the residue of a fatty acid comprising from 8 to 29 carbon atoms and R² represents an hydrocarbon chain, branched or unbranched, comprising from 3 to 30 carbon atoms, such as, for example, purcellin oil or 660084 PCL-LIQUID from Symrise (mixture of cetearyl 2-ethylhexanoate and of isopropyl myristate), isononyl isononanoate, isopropyl myristate, isopropyl palmitate, 2-ethylhexyl palmitate (or octyl palmitate), 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;
fatty alcohols having from 8 to 26 carbon atoms, such as isocetyl alcohol, isostearyl alcohol and their mixture (cetearyl alcohol), octyldodecanol, 2-butyloctanol, 2-hexyldecanol, 2-undecylpentadecanol, oleyl alcohol or linoleyl alcohol;
fluorinated oils partially hydrocarbonated and/or siliconated, such as those described in document JP-A-2-295912; mention may also be made, as fluorinated oils, of perfluoromethylcyclopentane and perfluoro-1,3-dimethylcyclohexane, which are sold under the names of “Flutec PC1®” and “Flutec PC3®” by BNFL Fluorochemicals; perfluoro-1,2-dimethylcyclobutane; perfluoroalkanes, such as dodecafluoropentane and tetradecafluorohexane, which are sold under the names of “PF 5050®” and “PF 5060®” by 3M, or bromoperfluorooctyl, sold under the name “Foralkyl®” by Atochem; nonafluoromethoxybutane, sold under the name “MSX 4518®” by 3M, and nonafluoroethoxyisobutane; or perfluoromorpholine derivatives, such as 4-(tri-fluoromethyl)perfluoromorpholine, sold under the name “PF 5052®” by 3M;
silicone oils, such as volatile or nonvolatile polymethylsiloxanes (PDMSs) comprising a linear or cyclic silicone chain which are liquid or pasty at ambient temperature, in particular cyclopolydimethylsiloxanes (cyclomethicones), such as cyclohexasiloxane and cyclopentasiloxane; polydimethylsiloxanes comprising alkyl, alkoxy or phenyl groups or alkyl, alkoxy or phenyl groups which are pendant or at the end of the silicone chain, which groups have from 2 to 24 carbon atoms; or phenylated silicones, such as phenyl trimethicones, phenyl dimethicones, phenyl(trimethylsiloxy)diphenylsiloxanes, diphenyl dimethicones, diphenyl(methyldiphenyl)trisiloxanes, (2-phenylethyl)trimethylsiloxysilicates and polymethylphenylsiloxanes;
their mixtures.

Use will preferably be made of volatile oils and in particular of non-oxidizable volatile oils, such as hydrocarbons, ethers or polydimethylsiloxane with a short chain (C₂-C₂₀). Mention may in particular be made of dicaprylyl ether and cyclopentasiloxane.

In a particular embodiment, the second composition of the assembly according to the invention is an anhydrous composition. In other words, the content of water in the second composition is low. Thus, the amount of water generally does not exceed 10% by weight, with respect to the total weight of the second composition. It can in particular comprise a small amount of water which generally originates from the starting materials used. The amount of water in the second composition is preferably less than or equal to 7% by weight, with respect to the total weight of the second composition, even more preferably less than 5% by weight, preferably less than 4% by weight.

In another embodiment, the second composition according to the invention is a composition which comprises water. The amount of water depends on the type of composition envisioned for the cosmetic care.

Preferably, this composition comprises water in an amount such that the mixture of the first and second compositions of the cosmetic assembly according to the invention comprises less than 70% by weight, preferably less than 50% by weight, of water, with respect to the total weight of the mixture, preferably less than 40% by weight. According to this form and preferably, the second composition comprises more than 10% by weight of water, with respect to the total weight of the second composition.

In a particular embodiment, the second composition of the assembly according to the invention does not comprise H₂O₂.

In another specific embodiment of the invention, the second composition is a composition comprising a zeolite as activator and an anhydrous or aqueous glycol vehicle comprising butylene glycol. Preferably, in this embodiment the glycol vehicle comprising butylene glycol is an aqueous vehicle.

In a specific embodiment, the compositions of the cosmetic assembly according to the invention can also comprise at least one gelling or thickening agent. A person skilled in the art will take care to incorporate, in one or the other of the compositions, or in both, any gelling agent compatible with a cosmetic or dermatological use. Mention may in particular be made, as gelling agent, of alkyl polyethylene glycols (or also known as alkyl PEGs), such as in particular PEG 120 methyl di(or tri)oleate (Glucamate DOE 120 from Noveon, for example) or PEG 150 pentaerythrityl tetrastearate (Crothix from Croda, for example); cellulose derivatives such as, in particular, hydroxypropyl methyl cellulose (Klucel HF Pharm from Aqualon Hercules, for example); poloxamers such as, in particular, a condensate of ethylene oxide, of propylene oxide and of ethylene oxide (such as, for example: 128 EO/54 PO/128 EO) (MW: 14 000), sold under the name Synperonic PE/F108 by Uniquema (INCI name: Poloxamer 338); or acrylic homo- and copolymers and homo- and copolymers of acrylamidomethylpropanesulfonic acid (AMPS: Ammonium Polyacryloyldimethyl Taurate (for example: Hostacerin AMPS from Clariant)).

The amount of gelling agents in the compositions can vary to a large extent; it is preferably between 0.01 and 10% by weight, preferably between 0.5 and 5% by weight, with respect to the total weight of the composition concerned.

It will also be possible to combine, in the compositions according to the invention, cosmetic or dermatological additives and/or active principles.

It can thus be advantageous to use foaming surfactants.

Foaming surfactants are detergents and differ from emulsifying surfactants in the value of their HLB (Hydrophilic Lipophilic Balance), the HLB being the ratio of the hydrophilic part to the lipophilic part in the molecule. The term “HLB” is well known to a person skilled in the art and is described, for example, in “The HLB system. A time-saving guide to Emulsifier Selection” (published by ICI Americas Inc, 1984).

For emulsifying surfactants, the HLB generally ranges from 3 to 8, for the preparation of water-in-oil (W/O) emulsions, and from 8 to 18, for the preparation of oil-in-water (O/W) emulsions, whereas foaming surfactants generally have an HLB of greater than 20.

Advantageously, the compositions according to the invention can comprise a surfactant having an HLB of greater than 20.

The surfactant can be present in a composition of the assembly according to the invention in an amount ranging from 0.1 to 50% by weight, preferably from 0.5 to 35% by weight, preferentially from 0.5 to 20% by weight, in particular from 1 to 15% by weight, indeed even from 5 to 10% by weight, with respect to the total weight of the composition concerned.

The nonionic surfactants can be chosen, for example, from alkylpolyglucosides (APGs), maltose esters, polyglycerolated fatty alcohols, glucamine derivatives, such as 2-ethylhexyloxycarbonyl-N-methylglucamine, and their mixtures. Use is preferably made, as alkylpolyglucosides, of those comprising an alkyl group comprising from 6 to 30 carbon atoms and preferably from 8 to 16 carbon atoms and comprising a hydrophilic (glucoside) group preferably comprising 1, 2 or 3 glucoside units. Mention may be made, as alkylpolyglucosides, for example, of decyl glucoside (Alkyl C9/C11 polyglucoside (1.4)), such as the product sold under the name Mydol 10® by Kao Chemicals, the product sold under the name Plantaren 2000 UP® by Cognis and the product sold under the name Oramix NS 10® by Seppic; caprylyl/capryl glucoside, such as the product sold under the name Oramix CG 110® by Seppic; lauryl glucoside, such as the products sold under the names Plantaren 1200 N® and Plantacare 1200® by Cognis; coco glucoside, such as the product sold under the name Plantacare 818/UP® by Cognis; and their mixtures.

The maltose derivatives are, for example, those described in document EP-A-566 438, such as 6′-(O-octanoyl)-D-maltose, or alternatively 6′-(O-dodecanoyl)-D-maltose, described in document FR-2 739 556.

Mention may be made, among polyglycerolated fatty alcohols, of polyglycerolated dodecanediol (3.5 mol of glycerol), a product sold under the name Chimexane NF® by Chimex.

Anionic surfactants can be chosen, for example, from soaps (alkali metal salts of fatty acids), carboxylates, acylamino acids, amidoether carboxylates, alkyl polyaminocarboxylates, alkyl ether sulfates such as sodium laureth sulfates, alkylsulfonates, isethionates, alkyl methyltaurates, alkyl sulfosuccinates, alkyl sulfoacetates, alkyl phosphates (mono- or dialkyl phosphates), their salts and their mixtures.

Mention may in particular be made, as carboxylates, of alkyl glycol carboxylic acids (or 2-(2-hydroxyalkyloxy)acetic acids) and their salts, such as, for example, sodium lauryl glycol carboxylate, sold under the names Beaulight SHAA® or Beaulight LCA-25N® by Sanyo (CTFA name: Sodium Lauryl Glycol Carboxylate), or its corresponding acid form, sold under the name Beaulight SHAA (Acid Form)® by Sanyo.

Mention may be made, as acylamino acids, for example, of sodium cocoyl glycinate, sold by Ajinomoto under the name Amilite GCS 12, sodium lauroyl glutamate, sold by Ajinomoto under the name Amisoft LS11, and sodium lauroyl sarcosinate, sold by Seppic under the name Oramix L 30.

Mention may be made, as alkyl phosphates, for example, of lauryl phosphate, sold by Kao under the name MAP 20.

Amphoteric and zwitterionic foaming surfactants can be chosen, for example, from betaine derivatives, including amidopropyl betaines, amphoacetates and amphodiacetates, hydroxysultaines and their mixtures.

Mention may be made, as betaine derivatives, for example, of coco betaine, such as the product sold under the name Dehyton AB-30® by Cognis; lauryl betaine, such as the product sold under the name Genagen KB® by Clariant; oxyethylenated lauryl betaine (10 OE), such as the product sold under the name Lauryl Ether (10 OE) Betaine® by Shin Nihon Rica; oxyethylenated stearyl betaine (10 OE), such as the product sold under the name Stearyl Ether (10 OE) Betaine® by Shin Nihon Rica; cocamidopropyl betaine, for example sold under the name Velvetex BK 35® by Cognis; undecylenamidopropyl betaine, for example sold under the name Amphoram U® by Ceca; and their mixtures.

Mention may be made, as amphoacetates and amphodiacetates, for example, of N-disodium N-cocoyl-N-carboxymethoxyethyl-N-(carboxymethyl)ethylene-diamine (CTFA name: disodium cocoamphodiacetate), such as the product sold under the name Miranol C2M Concentrate NP® by Rhodia Chimie; N-sodium N-cocoyl-N-hydroxyethyl-N-(carboxymethyl)ethylened iamine (CTFA name: sodium cocoamphoacetate), and their mixtures.

According to an embodiment of the invention, a surfactant suitable for the invention can be chosen from alkylpolyglucosides, betaine derivatives, alkyl glycol carboxylic acids and their salts, alkyl ether sulfates, alkyl phosphates, amphodiacetates, amphoacetates, alkylglycinates, acylglutamates, acylsarcosinates and mixtures of these.

According to an embodiment, a surfactant suitable for the invention can be chosen from decyl glucoside, cocoyl glucoside, sodium lauryl ether sulfate, cocoyl betaine, lauroyl betaine, cocoamidopropyl betaine, lauramidopropyl betaine, lauryl glycol carboxylate, cocoampho(di)acetate, lauroampho(di)acetate, potassium lauryl phosphate or a mixture of these.

Mention may be made, as preferred foaming surfactants, of:
sodium cocoyl isethionate, such as the sodium cocoyl isethionate/sodium isethionate mixture sold by BASF under the name Jordapon CI P®;
decyl glucoside, such as Plantacare 2000 UP® sold by Cognis;
potassium laurate, such as Laurate de Potassium DUB LK®, sold by Stearinerie Dubois;
sodium lauroyl OAT amino acids, such as the 30% aqueous solution of lauroyl oat amino acids, in the protected sodium salt form (1.4% of phenonip), sold under the name Proteol OAT® by Seppic;
potassium lauroyl wheat amino acids, such as stabilized (phenonip/potassium sorbate 0.4-0.4%) 25% in water potassium lauroyl hydrolyzed wheat protein, sold under the name Aminofoam W OR® by Croda;
polyglyceryl-3 hydroxylauryl ether or polyglycerolated dodecanediol (3.5 mol of glycerol), such as Chimexane® from Chimex;
sodium lauryl ether sulfate, such as Texapon AOS 225 UP® from Cognis;
N-disodium N-cocoyl-N-carboxymethoxyethyl-N-(carboxymethyl)ethylenediamine (CTFA name: disodium cocoamphodiacetate), such as the product sold under the name Miranol C2M Concentrate NP® by Rhodia Chimie;
coco betaine or cocoyl betaine in aqueous solution, such as Mirataine BB/FLA from Rhodia;
PEG-7 glyceryl cocoate or oxyethylenated glyceryl cocoate (70E), such as Cetiol HE® from Cognis;
lauryl betaine, such as the lauryl dimethylamine betaine in aqueous solution at 30% sold under the name Empigen BB/LS® by Huntsman;
sodium cocoamphoacetate, such as the sodium N-cocoylamidoethyl-N-(hydroxyethyl)glycinate as a 32% aqueous solution sold under the name Miranol Ultra C 32® by Rhodia;
triethanolamine lauryl sulfate or TEA-lauryl sulfate, such as the triethanolamine lauryl sulfate (C12/C14 70/30) as a 40% aqueous solution sold under the name Texapon T 42® by Cognis;
sodium lauryl glycol carboxylate, sold under the names Beaulight SHAA® or Beaulight LCA-25N® by Sanyo (CTFA name: Sodium Lauryl Glycol Carboxylate), or its corresponding acid form sold under the name Beaulight SHAA (Acid Form)® by Sanyo;
lauryl phosphate, sold by Kao under the name MAP 20®;
sodium cocoyl glycinate, such as Amilite GCS® 12 from Ajinomoto.

Use may also be made, in the compositions of the assembly according to the invention, of exfoliating particles, fillers or their mixtures.

Use may be made, as exfoliating particles, of exfoliating or scrubbing particles of mineral, vegetable or organic origin. Thus, use may be made, for example, of polyethylene beads or powder, such as those sold under the name Microthene MN 727 or Microthene MN 710-20 by Equistar or such as the powder sold under the name Gotalene 120 Colorless 2 by Dupont; Nylon particles, such as those sold by Arkema under the name Orgasol 2002 EXD NAT COS; fibers, such as polyamide fibers, such as those sold by Utexbel under the name Pulpe Polyamide 12185 Taille 0.3 mm; poly(vinyl chloride) powder; pumice (INCI name) such as pumice 3/B from Eyraud; ground fruit kernel shells, such as ground materials derived from apricot kernels or walnut shells; sawdust; glass beads; alumina (INCI name) (aluminum oxide), such as the product sold under the name Dermagrain 900 by Marketech International; sugar crystals; beads which melt during application to the skin, such as, for example, spheres based on mannitol and cellulose which are sold under the Unisphere names by Induchem, agar-based capsules which are sold under the Primasponge names by Cognis and spheres based on jojoba esters which are sold under the Florasphere names by Floratech; and their mixtures.

The compositions of the assembly according to the invention can also comprise other fillers, such as, for example, talc or modified or unmodified starch, in particular starches esterified with octenylsuccinic anhydride and more particularly “Aluminum Starch Octenylsuccinate”, such as the product sold by National Starch under the name of Dry-Flo.

According to an advantageous form, the compositions of the assembly according to the invention comprise at least one filler.

These fillers can be any material capable of masking visible or tactile irregularities of the skin. These fillers can in particular modify the surface appearance of the skin by a tightening effect, a concealing effect or a blurring effect.

The following compounds may be given as examples of filler:
porous silica microparticles, such as, for example, the Silica Beads® SB 150 and SB 700 from Myochi, with a mean size of 5 μm, and the Sunspheres® series H from Asahi Glass, such as H33 and H51, with a size respectively of 3.5 and 5 μm,
hollow hemispherical particles formed of silicone resins, such as NLK 500®, NLK 506® and NLK 510® from Takemoto Oil and Fat, in particular described in EP-A-1579849,
silicone resin powders, such as, for example, the Tospearl® 145 A silicone resin from GE Silicone, with a mean size of 4.5 μm,
powders formed of acrylic copolymers, in particular of polymethyl (meth)acrylate, such as, for example, the Jurimer MBI® PMMA particles from Nihon Junyoki, with a mean size of 8 μm, the hollow PMMA spheres sold under the name Covabead® LH 85 by Wackherr and the expanded vinylidene/acrylonitrile/methyl methacrylate microspheres sold under the name Expancel®,
wax powders, such as the Paraffin Wax Microease® 114S particles from Micropowders, with a mean size of 7 μm,
polyethylene powders, in particular comprising at least one ethylene/acrylic acid copolymer, such as, for example, Flobeads® EA 209 E from Sumimoto, with a mean size of 10 μm,
crosslinked organopolysiloxane elastomer powders coated with silicone resin, in particular with silsesquioxane resin, under the name KSP 100®, KSP 101®, KSP 102®, KSP 103®, KSP 104® and KSP 105® by Shin Etsu,
composite talc/dioxide of titanium/alumina/silica powders, such as, for example, Coverleaf AR 80® from Catalyst & Chemical,
talc, mica, kaolin, lauryl glycine, powders formed of starch crosslinked with octenylsuccinic anhydride, boron nitride, polytetrafluoroethylene powders, precipitated calcium carbonate, magnesium carbonate, basic magnesium carbonate, barium sulfate, hydroxyapatite, calcium silicate, cerium dioxide and glass or ceramic microcapsules,
hydrophilic or hydrophobic, synthetic or natural and inorganic or organic fibers, such as fibers formed of silk, of cotton, of wool, of flax, of extracted cellulose, in particular extracted from wood, vegetables or algae, of polyamide (Nylon®), of modified cellulose, of poly(p-phenylene terephthalamide), of acrylic, of polyolefin, of glass, of silica, of aramid, of carbon, of polytetrafluoroethylene (Teflon®), of insoluble collagen, of polyester, of polyvinyl chloride or polyvinylidene chloride, of polyvinyl alcohol, of polyacrylonitrile, of chitosan, of polyurethane or of polyethylene phthalate, fibers formed of a blend of polymers, resorbable synthetic fibers and their mixtures described in patent application EP 1 151 742,
spherical crosslinked silicone elastomers, such as Trefil E-505C® or E-506 C® from Dow Corning,
abrasive fillers which, by mechanical effect, contribute smoothing of the skin microrelief, such as abrasive silica, such as, for example, Abrasif SP® from Semanez, or powders formed of nuts or of shells (apricot, nuts, for example from Cosmétochem).

The filler can be a soft-focus filler. The term “soft-focus filler” is understood as a filler which, in addition, gives transparency to the complexion and a blurred effect. Preferably, the soft-focus fillers have a mean particle size of less than or equal to 15 microns. These particles can have any shape and can in particular be spherical or nonspherical. More preferably, these fillers are nonspherical.

The soft-focus fillers can be chosen from powders formed of silica and silicates, in particular of alumina, powders of polymethyl methacrylate (PMMA) type, talc, silica/TiO₂ or silica/zinc oxide composites, polyethylene powders, starch powders, powders formed of polyamides, powders formed of styrene/acrylic copolymers, silicone elastomers and their mixtures.

Mention may in particular be made of talc with a number-average size of less than or equal to 3 microns, for example talc with a number-average size of 1.8 microns, in particular that sold under the trade name Talc P3® by Nippon Talc, Nylon® 12 powder, in particular that sold under the name Orgasol 2002 Extra D Nat Cos® by Atochem, silica particles treated at the surface with 1 to 2% of a mineral wax (INCI name: Hydrated Silica (and) Paraffin), such as those sold by Degussa, amorphous silica microspheres, such as those sold under the name Sunsphere, for example with the reference H-53®, by Asahi Glass, and silica microbeads, such as those sold under the name SB-700® or SB-150® by Miyoshi, this list not being limiting.

These exfoliating particles and fillers can be present in an amount ranging, for example, from 0.5 to 20% by weight, preferably from 1 to 15% by weight, better still from 1 to 10% by weight and even better still from 2 to 5% by weight, with respect to the total weight of the composition.

The compositions of the assembly according to the invention can also comprise all the constituents normally employed in the application envisaged. In particular, advantageously, use may be made, in the compositions, of at least one additional ingredient and/or active principle for skin care, in particular for greasy skin.

It will be possible in particular to combine the additional active principles and ingredients described in patent application EP 1 847 247, incorporated here by reference, including depigmenting agents, preservatives, antimicrobial agents, antiperspirant agents, seboregulating agents, metal-chelating agents, UV filters, hydrolyzed proteins, antioxidants, vitamins, antiinflammatory agents, soothing or antiirritant agents, moisturizing agents, plant extracts, cosmetic adjuvants and their mixtures.

Of course, a person skilled in the art will take care to choose this or these optional additional compounds and/or their amounts so that the advantageous properties of the assembly according to the invention are not, or not substantially, detrimentally affected by the envisaged addition.

In an advantageous aspect of the invention, the compositions used can additionally comprise at least one desquamating agent and/or at least one soothing agent.

The term “desquamating agent” is understood to mean any compound capable of having an effect:
either directly on desquamation by promoting exfoliation, such as β-hydroxy acids, in particular salicylic acid and its derivatives (including 5-(n-octanoyl)salicylic acid); α-hydroxy acids, such as glycolic acid, citric acid, lactic acid, tartaric acid, malic acid or mandelic acid; urea; gentisic acid; oligofucoses; cinnamic acid; Saphora japonica extract; or resveratrol; for the acids, use is preferably made of their organic or inorganic salts; preferably, use is made of a salt of 5-(n-octanoyl)salicylic acid;
or on the enzymes involved in desquamation or decomposition of the corneodesmosomes, glycosidases, stratum corneum chymotryptic enzyme (SCCE) or indeed even other proteases (trypsin, chymotrypsin-like). Mention may be made of agents which chelate inorganic salts: EDTA; N-acyl-N,N′,N′-ethylenediaminetriacetic acid; aminosulfonic compounds and in particular N-(2-hydroxyethyl)piperazine-N′-2-ethanesulfonic acid (HEPES); 2-oxothiazolidine-4-carboxylic acid (procysteine) derivatives; derivatives of α-amino acids of glycine type (such as described in EP-0 852 949, and also sodium methylglycinediacetate, sold by BASF under the trade name Trilon M); honey; or sugar derivatives, such as 6-(O-octanoyl)-D-maltose and N-acetylglucosamine.

Mention may be made, as soothing agents which can be used in the compositions of the assembly according to the invention, of: pentacyclic triterpenes and the plant extracts (e.g.: Glycyrrhiza glabra) comprising them, such as β-glycyrrhetinic acid and its salts and/or its derivatives (glycyrrhetinic acid monoglucuronide, stearyl glycyrrhetinate, 3-stearoyloxyglycyrrhetic acid), ursolic acid and its salts, oleanolic acid and its salts, betulinic acid and its salts, a Paeonia suffruticosa and/or lactiflora extract, salicylic acid salts and in particular zinc salicylate, phycosaccharides from Codif, a Laminaria saccharina extract, canola oil, bisabolol and camomile extracts, allantoin, Sepivital EPC (phosphoric diester of vitamin E and C) from Seppic, omega-3 unsaturated oils, such as musk rose oil, blackcurrant oil, Echium oil or fish oil, plankton extracts, capryloyl glycine, Sepicalm VG (sodium palmitoyl proline and Nymphaea alba) from Seppic, a Pygeum extract, a Boswellia serrata extract, a Centipeda cunnighami extract, a Helianthus annuus extract, a Linum usitatissimum extract, tocotrienols, Cola nitida extracts, piperonal, a clove extract, an Epilobium angustifolium extract, aloe vera, a Bacopa moniera extract, phytosterols, cortisone, hydrocortisone, indomethacin and betamethasone.

These active principles will generally be present in the compositions of the assembly according to the invention in a content ranging from 0.01 to 20% by weight, preferably from 0.1 to 10% by weight, with respect to the total weight of the composition.

A person skilled in the art will take care to choose, from these active principles, those which can have an activity under the conditions of use of the compositions according to the invention. He will also take care to ensure that the active principles used in their amounts do not interfere with the advantageous properties of the compositions the constituting cosmetic assembly according to the invention, in particular in terms of pH.

Preferably, the active principles will be incorporated in the second composition of the cosmetic assembly, comprising the activator for the decomposition of H₂O₂, or in an additional composition of the cosmetic assembly.

The compositions of the assembly according to the invention can be provided in any galenic form normally used in the cosmetic or dermatological fields which is compatible with the characteristics of said compositions.

In particular, the first composition is composed of the polymer-H₂O₂ complex, preferably in the form of a powder, to be mixed with the second composition before application. In this context, the second composition comprises at least one activator and a vehicle which is acceptable cosmetically or dermatologically.

According to another alternative form, the first composition comprises at least one polymer-H₂O₂ complex and a vehicle which is acceptable cosmetically or dermatologically, which composition is mixed with the second composition. The second composition in this alternative form can be composed of one or more activators, advantageously in the powder form.

According to yet another alternative form, the first composition comprises at least one polymer-H₂O₂ complex and an anhydrous vehicle which is acceptable cosmetically or dermatologically, which is mixed with the second composition comprising at least one activator and a vehicle which is acceptable cosmetically or dermatologically, said vehicle of the second composition being anhydrous or aqueous.

The compositions of the cosmetic assembly of the invention can be more or less fluid and can have the appearance of a white or colored cream, of an ointment, of a milk, of a lotion, of a serum, of a paste or of a foam. They can optionally be applied to the skin in the form of an aerosol. They can also be provided in the solid form, for example in form of a stick. They can be used as care product and/or as makeup product.

According to the invention, the two compositions of the cosmetic assembly can be presented in various forms of cosmetic products, with various containers, compartments, closing elements, applicators, packagings, and the like.

The invention also relates to a packaging assembly comprising:
i. a first compartment comprising a first composition comprising a polymer-H₂O₂ complex,
ii. a second compartment comprising a second composition comprising at least one activator which promotes the decomposition of H₂O₂, said second compartment being isolated in a leaktight manner from the first, and
iii. means for making it possible to bring the two compositions into contact, in particular at the time of use.

The first composition and the second composition of the packaging assembly are as defined above.

Such an assembly advantageously makes it possible to bring its two compositions, packaged separately in respectively the first compartment and the second compartment forming said assembly, into contact at the time of use.

This assembly can also be provided with means which make it possible to bring the first compartment and the second compartment, and thus their respective contents, into contact.

The assembly is also advantageously provided with a means suitable for the dispensing of the mixture of the two compositions.

More specifically, the first composition and the second composition, for the implementation of the invention, are packaged separately inside two compartments, formed either by two separate containers, or inside a single device.

The container(s) can have any appropriate form. They can in particular be in the form of a bottle, a tube, a pot, a box, a tin, a bag or a case.

The closing element of the container(s) can be in the form of a removable stopper, of a lid, of a seal, of a tear-off strip or of a capsule, in particular of the type comprising a body fixed to the container and a cap articulated over the body. It can also be in the form of an element providing the selective closure of the container, in particular a pump, a valve or a flap.

The container can be used in combination with an applicator. The applicator can be in the form of a fine brush, such as described, for example, in patent FR 2 722 380. The applicator can be in the form of a pad of foam or elastomer, of a felt-tipped pen or of a spatula. The applicator can be free (powder puff or sponge) or integrally attached to a rod carried by the closing element, such as described, for example, in U.S. Pat. No. 5,492,426. The applicator can be integrally attached to the container, such as described, for example, in patent FR 2 761 959.

The compositions may be contained directly in the container or indirectly. By way of example, the compositions can be positioned on an impregnated support, particularly in the form of a wipe or of a wad, and can be positioned (single unit or several) in a tin or in a bag. Such a support incorporating the product is described, for example, in application WO 01/03538.

The closing element can be coupled to the container by screwing. Alternatively, the coupling between the closing element and the container is carried out by other means than by screwing, in particular via a bayonet mechanism, by snapping, clamping, welding or adhesive bonding, or by magnetic attraction. The term “snapping” is understood to mean in particular any system involving the crossing of a roll or strip of material by elastic deformation of a portion, in particular of the closing element, and then by elastically returning said portion to the unstressed position after the roll or strip has been crossed.

The container can be at least partially made of thermoplastic material. Mention may be made, as examples of thermoplastic materials, of polypropylene or polyethylene.

Alternatively, the container is made of nonthermoplastic material, in particular of glass or of metal (or alloy).

The container can have rigid walls or deformable walls, in particular can be in the form of a tube or of a tube bottle.

The container can comprise means intended to bring about or facilitate the dispensing of the composition. By way of example, the container can have deformable walls, so as to bring about squeezing of the composition in response to excess pressurization inside the container, which excess pressurization is brought about by the elastic (or nonelastic) crushing of the walls of the container.

The container can be composed of a case with a bottom delimiting at least one receptacle comprising the composition and a lid, in particular articulated over the bottom, capable of at least partially covering said bottom. Such a case is described, for example, in patent application WO 03/018423 or in patent FR 2 791 042.

The container can be equipped with a drainer positioned in the vicinity of the opening of the container. Such a drainer makes it possible to wipe the applicator and optionally the rod to which it may be integrally attached. Such a drainer is described, for example, in patent FR 2 792 618.

According to a specific form of the invention, the container is in the form of a bottle or a flexible bag.

The term “single device” is understood to mean a device via which the two compartments or containers are integrally attached to one another. Such a device can be obtained by a process of molding the two compartments, in particular made of a thermoplastic material, as a single component. It can also result from any form of assembling, in particular by adhesive bonding, welding or other snapping operation.

According to a first embodiment, the two containers are separate, independent of one another. Such containers can be provided in various forms. They can in particular be tubes, bottles or cans.

One and/or the other of the containers can be surmounted by a manually actuated pump surmounted by a pushbutton for the actuating of the pump and the dispensing of the composition via at least one dispensing orifice.

Alternatively, one and/or the other of the containers are pressurized, in particular using a propellant, in particular a propellant gas. In this case, the container(s) is (are) equipped with a valve surmounted by a pushbutton equipped with a nozzle or any other diffusion means for the dispensing of the product.

The propellant can be as a mixture with at least one of the compositions to be dispensed or can be separated, in particular via a piston capable of sliding inside the container or via the flexible walls of a pocket inside which the composition is positioned.

The containers can be composed of various materials: plastic, glass or metal.

According to a preferred embodiment, the two compositions are present inside a single device.

According to a specific form, the first composition and the second composition of the invention are packaged inside two compartments (51, 52) delimited by a single device (1) of pump-action spray type as represented in FIG. 1 of U.S. Pat. No. 6,672,483.

In particular, each of the compartments is equipped with a pump (41, 42), preferably manually actuated, connected to at least one actuating and dispensing means (3) which makes it possible to deliver, separately or as a mixture, said first composition and second compositions.

According to a preferred form, the actuating and dispensing means (3) is common to the two pumps.

According to one alternative, each of the compartments is pressurized, in particular using a propellant, and is equipped with a valve connected to at least one actuating and dispensing means which makes it possible to deliver, separately or as a mixture, said compositions.

In particular, the actuating and dispensing means is common to the two valves.

The packaging device presented in FIG. 1 of U.S. Pat. No. 6,672,483 is composed of two compartments 51, 52 positioned side by side and formed inside a component 5 obtained by molding a thermoplastic material. Each of the containers 51, 52 comprises a neck 53 delimiting an opening. A pump 41, 42, which may or may not have air reuptake, is mounted inside the neck of each of the containers.

During the mounting, component 5 delimiting the two compartments 51, 52 is positioned inside a housing element 10.

A pump shaft 21a, 22a of each of the pumps 41, 42 is inserted forcibly inside a corresponding pipe provided in a single pushbutton 3 configured so as to make possible the simultaneous actuating of the two pumps in response to a pressure exerted axially on a surface 35 of pushbutton 3.

The pipes of the pushbutton connected to each of the pumps emerge on two orifices 31a, 32a positioned in the vicinity of one another on an external surface of pushbutton 3. The two compositions exit separately in response to an actuation of the pumps 41, 42, either on the finger of the user or on an applicator wad or swab. The mixing of the two compositions then takes place during application to the surface to be treated.

Use may be made, for example, of devices as described in the documents U.S. Pat. No. 5,833,121, U.S. Pat. No. 4,773,562 and U.S. Pat. No. 6,672,483.

According to another specific form, the two compartments are formed inside the same flexible bag and are separated by a weak weld or tearing region which can be broken during use, in particular in response to a pressure exerted at a precise point of the bag, at one of the two compartments. Examples of two-compartment flexible bags which can be used in the present invention are described in particular in patent applications JP2000272674, WO99/51509, EP 2 437 30 or FR 2 876 356. The compartments can be produced by means of at least one sheet, for example a sheet of a flexible thermoplastic material, and can be bonded to one another via a common edge of weak weld type. According to a specific form, the common edge of weak weld type is over the entire width of the bag or over a lesser width forming a leaktight channel between the two bags.

Preferably, the bag is produced with films impermeable to air oxygen, such as aluminum.

Use may also be made of films impermeable to water in the form of a polyethylene terephthalate/silicone/polyethylene (PET/SiOx/PE) triple layer.

Alternatively again, the two compartments are formed of two concentric compartments formed inside a tube and are surmounted by a pump without air reuptake equipped with a pushbutton having one or two dispensing orifices. A piston is provided inside the tube, which piston rises up in the direction of the pump as the compositions are withdrawn from inside the containers. Such dispensing modes are used in particular for the dispensing of toothpastes.

The contents of the abovementioned patents or patent applications are incorporated by reference in the present application.

Yet other devices can be used for the implementation of the present invention, the essential point being that they can make possible the separate packaging of the two compositions of the cosmetic assembly of the present invention and the dispensing thereof separately or as a mixture.

The compositions of the cosmetic assembly according to the invention, after having been mixed, can form a mask, a cleansing, protecting, treating or care cream for the face, for the hands, for the feet or for the body (for example day creams, night creams, makeup-removing creams, foundation creams or antisun creams), a makeup-removing milk, or a lotion, gel or foam for caring for the skin, such as a cleansing lotion.

The cosmetic assembly according to the invention has in particular a lightening effect and a positive effect on the radiance of the complexion, it makes it possible to tone down age spots and/or it has a positive effect on the appearance of greasy skin, in particular by promoting the mattness of the skin. The cosmetic assembly according to the invention also makes it possible to lighten the skin, to promote the radiance of the complexion, to render the complexion uniform, to reduce spots, in particular age spots, or to improve the hold of the makeup.

Consequently, the invention relates to a cosmetic method for improving the appearance of the skin, comprising the application, to the skin, of the cosmetic assembly according to the invention.

More specifically, the invention relates to a method for softening the imperfections of the skin, the spots and/or the acne lesions, for smoothing the skin relief, for rendering the pores less apparent (in particular by reducing the size of the pores), for rendering the microrelief and/or the wrinkles less visible, for restoring radiance to the skin, in particular dull, ashen or dark skin, for improving the appearance of greasy skin, in particular by promoting the mattness of the skin, for lightening the skin, for promoting the radiance of the complexion, for rendering the complexion uniform, for reducing spots, in particular age spots or spots due to the sun, and/or for improving hold of the makeup, comprising the application, to the skin, of the assembly according to the invention.

Softening of the imperfections of the skin will also take place by reducing the number of pimples and blackheads and/or reducing the shininess of the skin.

The assembly according to the invention can be applied in all the ways which can be envisaged by a person skilled in the art in a method for caring for the skin.

In particular, it can be envisaged to mix the compositions of said assembly before application to the skin. The properties of the polymer present in the first composition, in particular PVP, make possible easy application of the assembly according to the invention after mixing the compositions constituting it, the mixture not flowing or flowing only slightly over the skin after application. Alternatively, it is possible to envisage the prior application of one of the compositions to the skin, followed by the application of the other composition to the skin thus prepared.

For example, in a particular embodiment, the first composition of the assembly according to the invention, comprising the polymer-H₂O₂ complex, preferably a PVP-H₂O₂ complex, is first applied to the skin. Subsequently, the second composition, comprising the activator which promotes the decomposition of H₂O₂, is applied to the skin, at least over the area where the first composition was applied.

In another embodiment, the second composition, comprising the activator defined above, is first applied and then the first composition, comprising the polymer-H₂O₂ complex, is applied to the skin, at least over the area where the second composition, comprising the activator, was applied.

According to a preferred form, said method according to the invention additionally comprises a rinsing stage, after application of the cosmetic assembly to the skin or keratinous appendages.

According to a specific embodiment, the cosmetic method according to the invention comprises
mixing the first and second compositions of the cosmetic assembly according to the invention with water, the first composition comprising a PVP-H₂O₂ complex and a glycol vehicle comprising PEG-8 and the second composition being composed of a zeolite in the powder form, and
application of the resulting mixture to the skin.

A further object of the invention is the cosmetic use of the cosmetic assembly as described above for improving the appearance of the skin and more specifically for softening the skin imperfections, the spots and/or the acne lesions, for smoothing the skin relief, for rendering the pores less apparent (in particular by reducing the size of the pores), for reducing the number of pimples and blackheads, microrelief and/or the wrinkles less visible, for restoring radiance to the skin, in particular dull, ashen or dark skin, for improving the appearance of greasy skin, in particular by promoting the mattness of the skin or by reducing the shininess of the skin, for lightening the skin, for promoting the radiance of the complexion, for rendering the complexion uniform, for reducing spots, in particular age spots or spots due to the sun, and/or for improving the hold of the makeup, comprising the application, to the skin, of the cosmetic assembly according to the invention as described above.

The cosmetic assembly according to the invention also has the advantage of being able to make possible the lightening of keratinous appendages, in particular of the head hair or body hair or downy hair, on the forearms, on the face, in particular at the moustache, and the like.

Another object of the invention is a cosmetic method for caring for greasy skin, in particular formattifying the skin, comprising the topical application thereon of the cosmetic assembly as described above.

According to a preferred embodiment, said cosmetic method for caring for greasy skin additionally comprises a rinsing stage after application to the skin of the compositions of the cosmetic assembly as described above.

In particular, the cosmetic assembly is applied to shiny areas of the face or forehead.

According to a particular form, the skin can be wetted prior to the application of the cosmetic assembly according to the invention.

The methods according to the invention are more particularly intended for subjects having skin of phototype III to VI.

According to a preferred form, after application on the skin, the mixture of the compositions of the cosmetic assembly is rinsed off, after a leave-on time advantageously of between 5 and 30 minutes, preferably after a leave-on time of less than 20 minutes, even more preferably for a time of approximately 15 minutes.

The cosmetic assembly can be used as a mask, preferably with a weekly, twice monthly or monthly frequency of application. It can also be used as a course of treatment with, for example, a daily or twice daily application for one or two weeks.

The invention relates in addition to an assembly as described above as a product for the treatment of acne.

The invention thus also relates to the use of an assembly as described above in the manufacture of a product for the treatment of acne.

According to another aspect, the assembly according to the invention is used as an antibacterial active principle for reducing the amount of germs responsible for acne present on the skin, or for limiting their growth, and in particular for reducing the amount of P. acnes, or for limiting the growth of one of these germs.

The term “treatment” and all its declensions are intended to denote the curative or preventive treatment. The assembly according to the invention can thus be used in a subject with acne. The assembly according to the invention can also be used to delay or slow down the progression or to prevent a more advanced progression of acne. Finally, the assembly according to the invention can be administered to a person not exhibiting acne but whose skin, in particular greasy skin, has a tendency toward acne.

The amount of active principles present in the compositions of the assembly according to the invention will advantageously be within the intervals described above, in order to obtain the desired effect. Of course, a person skilled in the art is in a position to establish the effective amount of said active principles in the assembly according to the invention and will take care that the additional ingredients and/or active principles introduced into the first and second composition described above do not detrimentally affect the advantageous effects of the assembly according to the invention on the decrease in the germs of acne.

According to another aspect, the invention relates to a method for the dermatological treatment of acne, comprising the topical administration of the assembly according to the invention described above, after mixing the first and second compositions as described above, to a person in need thereof.

The composition can be applied according to any way which can be envisaged by a person skilled in the art and in particular according to the methods described above for the cosmetic methods according to the invention.

The examples which follow illustrate the invention without limiting the scope thereof. The compounds are, as the case may be, mentioned as chemical names or as CTFA (International Cosmetic Ingredient Dictionary and Handbook) names.

### LEGENDS TO THE FIGURES

FIG. 1 is a graph demonstrating the activation of a polymer-H₂O₂ complex. This FIGURE presents the ratio between the level of dissolved oxygen at a time t and the level of oxygen at t₀ as a function of time.

### EXAMPLES

### Example 1

### Lightening Activity Measured In Vitro

The lightening activity of a commercial PVP-H₂O₂ complex, Peroxydone K-30® from ISP, is measured in various solvents in a bleaching test on a dye.

Protocol for in vitro bleaching test Dye solution or “solution X” is a solution of disodium salt of brilliant blue FCF (CI: 42090) from LCW (Sensient). The dye solution is prepared as follows: 15 mg of dye powder are dissolved in 50 ml of borate buffer pH 10.7 and then again diluted to 1/50^{th}. The borate buffer is prepared in the following way: 4.77 g of disodium tetraborate decahydrate (Na₂B₄O₇ 10H₂O, M=381.37 g/mol) are dissolved with approximately 500 ml of water. pH is adjusted to 10.7 by addition of a sodium hydroxide solution (250 ml approximately of 0.1N NaOH made up to 1 liter).

1 g of a 22% dispersion of Peroxydone K-30® in the dilution medium under consideration (ethanol, PEG-8, butylene glycol or glycerol) is added to 19 g of solution X. After stirring the mixture in the dark for 10 min, the dispersion is filtered (0.45 μm cellulose filter) and the optical density is measured over the range from 400 to 800 nm at 25° C. The dye absorbs at 630 nm. The decoloring effectiveness is reflected by the bleaching percentage at t=10 min (i.e. [OD_{t10 min} (mixture)−OD_{t10 min} (dye)/ODₜ₀ (dye)]×100.

The results are given in the following table:

| | Dilution medium | | | |
|---|---|---|---|---|
| | | | Butylene | |
| H₂O₂ source | Ethanol | PEG-8 | glycol | Glycerol |
| H₂O₂, 4% | 72% | 61% | 63% | 27% |
| PVP-H₂O₂, 22% | 75% | 69% | 50% | 30% |
| (eq. 4% H₂O₂) | | | | |

It is found that the PVP-H₂O₂ exhibits the same activity as free hydrogen peroxide, at an equivalent level of H₂O₂, whatever the formulation medium is, and is particularly effective in ethanol and PEG-8. The results obtained in butylene glycol are also satisfactory. These solvents will be preferred to glycerol.

### Example 2

### Formulation Examples

Preparations P1 and P2 shown in the table below are mixed before application to the skin.

20 g of formulation are applied as a thick layer to the face while avoiding the outline of the eyes. The skin can optionally be moistened before application of the mixture. The product is left for 10 min and then rinsed off.

| | Example 2a | | Example 2b | | Example 2c | | Example 2d | | Example 2e | | Example 2f | |
|---|---|---|---|---|---|---|---|---|---|---|---|---|
| | P1 + P2: Gel | | P1 + P2: Gel | | P1 + P2: Gel | | P1 + P2: Gel | | P1 + P2: Gel | | P1+ P2: Gel | |
| | comprising | | comprising | | comprising | | comprising | | comprising | | comprising | |
| Nature of the | 2% eq. H₂O₂ | | 2% eq. H₂O₂ | | 2% eq. H₂O₂ | | 2.44% eq. | | 2% eq. H₂O₂ | | 4% eq. H₂O₂ | |
| mixture | pH 9.5 | | pH 10.1 | | pH 9.1 | | H₂O₂ pH 10 | | pH 10.1 | | pH 10 | |
| Weight (g) | P1 | P2 | P1 | P2 | P1 | P2 | P1 | P2 | P1 | P2 | P1 | P2 |
| Water | | | | | | 44.6 | | | | 27 | | 40 |
| Peroxydone K- | 11 | | 11 | | 11 | | 11 | | 11 | | 22 | |
| 30 ® (18% eq. | | | | | | | | | | | | |
| H₂O₂) | | | | | | | | | | | | |
| Butylene glycol | | | 50 | | | | | | | | | |
| PEG-8 | 39 | | | | 20 | | 45 | | | 27 | | 37.4 |
| Absolute | 15 | | 38.65 | | 19 | | 40 | | | | | |
| ethanol | | | | | | | | | | | | |
| Laponite | | | | | | 5.4 | | | | | | |
| Zeolite | | 35 | | | | | | | | 35 | | |
| Sodium | | | | 0.35 | | | | | | | | 0.6 |
| metasilicate | | | | | | | | | | | | |
| Calcium | | | | | | | | 4 | | | | |
| peroxide (14% | | | | | | | | | | | | |
| eq. H₂O₂) | | | | | | | | | | | | |

Note:
Peroxydone K-30 ® is a PVP-H₂O₂ complex sold by ISP; the calcium peroxide used is sold by SPCH.

The mixtures are easy to apply (do not flow) due to the film-forming nature of PVP-H₂O₂.

### Example 3

### Clinical Test of Effectiveness on Greasy Skin

The two products A and B shown in the table below were tested on 40 persons having greasy skin with a tendency toward acne (presence of pimples/blackheads) with dilated pores according to a monocentric, randomized, double blind and half-face protocol.

After mixing the preparations of P1 and P2 (for each of the two products) in a 1.5 g/1.5 g ratio in a small dish, products A and B were applied (at a amount of 3 g) as a thick layer to one side of the moistened face every day for 5 days (one product per cheek for the same person). The leave-on time was 10 minutes. The product was subsequently rinsed off thoroughly.

| | Product A | | Product B (Placebo) | |
|---|---|---|---|---|
| Amount | Preparation 1 | Preparation 2 | Preparation 1 | Preparation 2 |
| in g (%) | (P1) | (P2) | (P1) | (P2) |
| Peroxydone | 26 | | | |
| K-30 ®* | | | | |
| (eq. 2.3% | | | | |
| H₂O₂) | | | | |
| PVP | | | 20.7 | |
| K30L ®** | | | | |
| PEG-8 | q.s. for 100 | | q.s. for 100 | |
| Zeolite*** | | 20 | | 20 |
| Butylene glycol | | 40 | | 40 |
| Carbomer | | 3 | | 3 |
| Sodium | | 0.078 | | 0.078 |
| hydroxide | | | | |
| Water | | q.s. for 100 | | q.s. for 100 |

*Peroxydone K-30 ® is a PVP-H₂O₂ complex sold by ISP which comprises 20% of H₂O₂ and 80% of PVP K30 L
**Polyvinylpyrrolidone sold by ISP
***X-MOL (Zeochem)

Preparation 1 is obtained by dispersion of Peroxydone K-30® in PEG-8 using a paddle until a transparent gel is obtained.

Preparation 2 is obtained by dispersion of the zeolite powder using a Rayneri mixer, after having caused the carbomer to swell in a water/sodium hydroxide mixture and having introduced the butylene glycol.

The values measured are the scoring (size) of the pores (Dermascore) and the surface area of the porphyrins (marker of activity on the bacterium Propionobacterium acnes).

After 5 days of daily application, a significant superiority of the treatment for product A appeared in comparison with the placebo (product B) as regards:
the reduction in the size of the pores,
the reduction in the surface area of the porphyrins (equivalent to a reduction in the activity of P. acnes germs responsible for acne), evidence of an antibacterial effectiveness.

### Example 4

### Demonstration of the Activation of the Polymer-H₂O, Complex

Product A is prepared as in example 3 above and product C is prepared in an identical fashion to the product A with the exclusion of the sodium hydroxide and the zeolite in the preparation P2.

| | Product A (basic pH) | | Product C (acidic pH) | |
|---|---|---|---|---|
| Amount | Preparation 1 | Preparation 2 | Preparation 1 | Preparation 2 |
| in g (%) | (P1) | (P2) | (P1) | (P2) |
| Peroxydone | 26 | | 26 | |
| K-30 ®* | | | | |
| (eq. 2.3% | | | | |
| H₂O₂) | | | | |
| PEG-8 | q.s. for 100 | | q.s. for 100 | |
| Zeolite** | | 20 | | 20 |
| Butylene glycol | | 40 | | 40 |
| Carbomer | | 3 | | 3 |
| Sodium | | 0.078 | | |
| hydroxide | | | | |
| Water | | q.s. for 100 | | q.s. for 100 |

| | |
|---|---|
| pH of the mixture = 8.3 | pH of the mixture = 4.2 |

*Peroxydone K-30 ® is a PVP-H₂O₂ complex sold by ISP which comprises 20% of H₂O₂ and 80% of PVP K30 L
**X-MOL (Zeochem)

The dissolved oxygen is quantitatively determined with the model 782 oxygen meter [Strathkelvin Instruments] connected to a microcathode oxygen electrode (model 1302).

Preparation for the formulation A: P1 is diluted to 10% in water. Likewise for P2. 500 μl of each dilute solution are introduced into a chamber with a regulated temperature, the MT200 Mitocell [Strathkelvin Instruments], in which the electrode is present. The dissolved oxygen is quantitatively determined, after a contact time of 10 minutes, in μg of dissolved oxygen/ml. The same operation is carried out for product C.

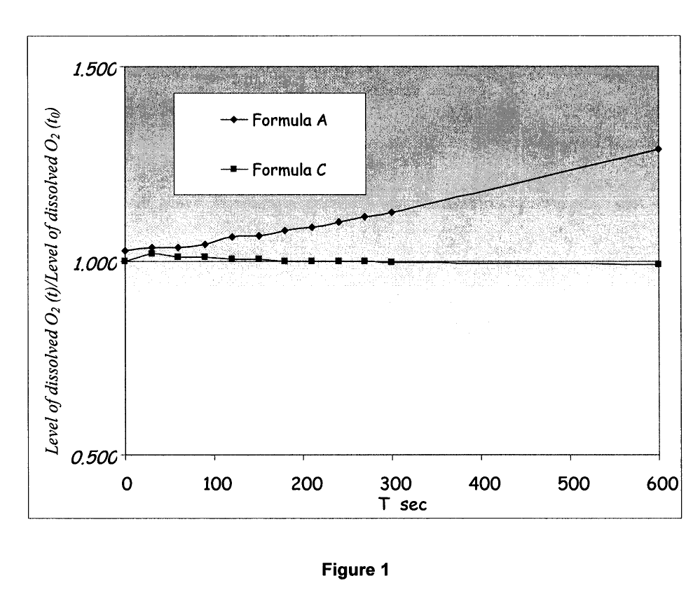

The results obtained are represented in FIG. 1 in the form of a ratio of the concentration of oxygen at the measurement time (t) to the concentration of dissolved oxygen measured at the beginning of the experiment (tₒ) as a function of the time.

An excess of dissolved oxygen (at least 25% greater) is thus measured for product A (corresponding to formulation A in FIG. 1) in comparison with product C (corresponding to formulation C in FIG. 1).

Formation of dissolved oxygen is found for product A and not for product C.

The germs responsible for acne (P. acnes) grow in the absence of oxygen (anaerobic bacterium); it is thus shown that the PVP-H₂O₂ complex provides a better activity than in its nonactivated form.

### Example 5

### Antibacterial Activity of the PVP-H₂O₂ Complex

The antibacterial activity of product A according to example 3 against the bacterial strain Propionibacterium acnes was determined.

This strain was brought into contact with product A in a liquid culture medium in the following ratios:
10% of the bacterial inoculum comprising 10⁸ microorganisms/ml,
10% of product A,
80% of liquid culture medium.

At the same time, a growth control in which product A was replaced, on the one hand, with diluent (water) or, on the other hand, with product B according to example 3 was prepared under the same conditions.

The samples were placed in a water bath at 35° C. and kept shaken throughout the duration of the test.

P. acnes bacteria living after 2 h of contact are counted in numbers/ml.

| | | Product A |
|---|---|---|
| | Growth control | (eq. 2.3% H₂O₂) |
| t 0 | 2.40E+07 | 2.40E+07 |
| t 15 min | 2.40E+07 | 8.60E+05 |
| t 30 min | 2.60E+07 | 1.60E+03 |
| t 1 hr | 2.30E+07 | 10 |

| | Product A | Product B |
|---|---|---|
| | (eq. 2.3% H₂O₂) | (eq. 0% H₂O₂) |
| t 0 | 2.10E+07 | 2.10E+07 |
| t 2 h | 2.10E+07 | 10 |
| t 6 h | 2.10E+07 | 10 |

The above tables show that product A based on the PVP-H₂O₂ complex (Peroxydone K-30® comprising an equivalent level of 2.3% H₂O₂) indeed has an antibacterial activity against Propionibacterium acnes.

1. A cosmetic assembly comprising:
a first anhydrous composition comprising at least one polymer-H₂O₂ complex, said polymer having hydrogen-acceptor groups which are capable of forming a noncovalent chemical bond with hydrogen peroxide, and
a second composition comprising at least one activator which promotes the decomposition of H₂O₂.

2. The assembly according to claim 1, wherein the polymer of the polymer-H₂O₂ complex is a polymer comprising N-vinylheterocyclic units.

3. The assembly according to claim 1 wherein the first composition comprises the polymer-H₂O₂ complex, and
the first composition is a powder.

4. The assembly according to claim 1 wherein the first composition further comprises an anhydrous cosmetically or dermatologically acceptable vehicle.

5. The assembly according to claim 1 wherein
the second composition consists of one or more activators, and
the second composition being in particular in the form of a powder.

6. The assembly according to claim 1 wherein the second composition comprises at least one activator which promotes the decomposition of H₂O₂ and an anhydrous cosmetically or dermatologically acceptable vehicle.

7. The assembly according to claim 1, wherein the activator is a compound which generates a pH greater than 8.5 when mixed with a polymer-H₂O₂ complex at 11% by weight in water, with respect to the total weight of the polymer-H₂O₂/water mixture.

8. The assembly according to claim 1, wherein the activator which promotes the decomposition of H₂O₂ is selected from group consisting of sodium bicarbonate, laponite, zeolites, calcium peroxide, sodium hydroxide, sodium metasilicate and a mixture thereof.

9. The assembly according to claim 4 wherein the anhydrous vehicle of the first composition is an alcohol, a glycol or a mixture thereof.

10. The assembly according to claim 1, wherein an amount of peroxide in the first composition is between 0.1 and 30, with respect to the total weight of the cosmetic assembly.

11. The cosmetic assembly according to claim 1, wherein the polymer-H₂O₂ complex is a PVP-H₂O₂ complex and the activator is a zeolite.

12. The cosmetic assembly according to claim 1, wherein
the first composition comprises a PVP-H₂O₂ complex and a glycol comprising PEG-8 and
the second composition comprises a zeolite.

13. A packaging assembly comprising:
i. a first compartment comprising a first composition comprising a polymer-H₂O₂ complex,
ii. a second compartment comprising a second composition comprising at least one activator which promotes the decomposition of H₂O₂, said second compartment being isolated in a leaktight manner from the first, and
iii. a dispensed which brings the two compositions into contact, at the time of use,
wherein
the first composition and the second composition of the packaging combination defined in claim 1.

14. A cosmetic method for improving the appearance of the skin, comprising the application, to the skin, of the cosmetic assembly according to claim 1 wherein improving the appearance of the skin comprises at least one selected from the group consisting of a lightening effect on the skin, a positive effect on the radiance of a complexion, a toning down of age spots and a promotion of a mattness of the skin.

15. The method according to claim 14, wherein the improvement in the appearance of the skin comprises at least one selected from the group consisting of softening of skin imperfections, spots and/or acne lesions, smoothing of skin relief, obtaining pores which are less apparent, obtaining a microrelief and/or wrinkles which are less visible, improvement in the radiance of the skin, promoting mattness of the skin, lightening of the skin, improvement in the uniformity of the complexion, reduction in age spots or spots due to the sun, and improvement in hold of the makeup.

16. A cosmetic method for lightening keratinous appendages, comprising topical application thereon, of the cosmetic assembly according to claim 1.

17. The method according to claim 14, further comprising rinsing the skin or keratinous appendages, after application of the cosmetic assembly to the skin or its keratinous appendages.
